(19) United States
(12) Patent Application Publication
(10) Pub. No.: US 2010/0066867 A1
(43) Pub. Date: Mar. 18, 2010

(54) IMAGING SYSTEM AND SEMICONDUCTOR INTEGRATED CIRCUIT

(76) Inventor: Hiroki YOSHIKAWA, Kyoto (JP)

Correspondence Address: MCDERMOTT WILL & EMERY LLP, 600 13TH STREET, NW, WASHINGTON, DC, 20005-3096, US

(21) Appl. No.: 12/525,629

(22) PCT Filed: Nov. 18, 2008
(86) PCT No.: PCT/JP2008/003368

§ 371 Date: Aug. 3, 2009

(30) Foreign Application Priority Data
Dec. 26, 2007 (JP) 2007-333725

(51) Int. Cl. H04N 5/217; H04N 5/335

(52) U.S. Cl. 348/241; 348/294; 348/E05.079; 348/E05.091

(57) ABSTRACT

An imaging system includes a solid-state imaging device (101) configured to convert light into an electrical signal to output a captured-image signal, an AFE unit (107) configured to process the captured-image signal from the solid-state imaging device, an image acquiring unit (110) configured to acquire an image having a specific color from the AFE unit (107), a noise detector (112) configured to detect noise of the specific-color image acquired by the image acquiring unit (110), and an AFE power-down controller (111) configured to control timing of power supply to the AFE unit (107) during a horizontal or vertical blanking period based on an amount of the noise detected by the noise detector.

### TECHNICAL FIELD

The present invention relates to a technique of controlling timing of supplying power to an Analog Front-End (AFE), which is an analog processing circuit for converting an analog captured-image signal from a solid-state imaging device into a digital signal.

### BACKGROUND ART

In recent years, a capsule-shaped camera (hereinafter referred to as a capsule camera), which is a medical camera apparatus for taking an image or a photograph in the medical field, has been put into practical use as a gastrocamera or the like so as to reduce the impact on the user. It is desirable that the capsule camera should have a small geometrical size, low power consumption and high image quality. In order to reduce a geometrical size, it is necessary to reduce a size of an imaging device or a process. In order to suppress power consumption, it is necessary to stop supply of power to a portion which is not being used. In this case, a technique of stopping power supply to an AFE during a blanking period is often utilized. This technique can reduce power consumption without impairing image quality.

Note that prior art documents relating to the present invention include Patent Documents 1 and 2. Patent Document 1 discloses a method of using dummy data to reduce deterioration in image quality due to the influence of noise on an analog circuit when valid data and invalid data are both present. Patent Document 2 discloses a method of suppressing the influence of noise entering from a power source, an external circuit or the like into a clamp circuit for clamping an output signal during a black reference signal period in an analog front-end IC chip having a CDS function, an AGC function or the like. These prior art documents are mainly directed to an improvement in image quality, and do not mention an improvement in power consumption.

### Patent Document 1: Japanese Unexamined Patent Application Publication No. 2007-189391

### Patent Document 2: Japanese Unexamined Patent Application Publication No. 2003-163845

### DISCLOSURE OF THE INVENTION

### Problems to be Solved by the Invention

In the aforementioned methods, however, when power supply is interrupted during a blanking period (the AFE is powered down), a waveform distortion occurs at rising when power supply is subsequently resumed. In this case, an input image may be contaminated with low-frequency noise (particularly, a black image is significantly affected). The contamination with low-frequency noise can be suppressed by previously adjusting the timing of powering down the AFE. However, the distortion may be changed by a disturbance, such as a change in temperature or the like, so that noise may occur again at a position where the adjustment has been performed (dynamic adjustment is not provided).

An object of the present invention is to provide an imaging system capable of obtaining an image having less noise and high image quality and reducing power consumption.

### Solution to the Problems

An imaging system according to the present invention includes a solid-state imaging device configured to convert light into an electrical signal to output a captured-image signal, an analog front-end (AFE) unit configured to process the captured-image signal from the solid-state imaging device, an image acquiring unit configured to acquire an image having a specific color from the AFE unit, a noise detector configured to detect noise of the specific-color image acquired by the image acquiring unit, and an AFE power-down controller configured to control timing of power supply to the AFE unit during a horizontal or vertical blanking period based on an amount of the noise detected by the noise detector.

According to the imaging system, an image having less noise and high image quality can be obtained, and power consumption can be reduced.

Also, in the imaging system, the image acquiring unit can acquire the specific-color image in any frame of the captured-image signal from the solid-state imaging device. The image acquiring unit adjusts a frequency of acquiring the specific-color image, depending on the amount of the noise detected by the noise detector.

According to the imaging system, the user can reduce a time required to obtain an image without noise.

Also, in the imaging system, the noise detector determines the amount of the noise based on an average value of luminance of the specific-color image acquired by the image acquiring unit.

Also, in the imaging system, the noise detector determines the amount of the noise based on a variance value of luminance of the specific-color image acquired by the image acquiring unit.

Also, in the imaging system, the noise detector detects noise from a predetermined horizontal line.

According to the imaging system, not all pieces of pixel data in a frame need to be used. Therefore, the detection speed can be improved.

Also, the imaging system further includes a vertical streak correction switching unit configured to switch on/off a process (vertical streak correction process) of removing noise from the captured-image signal output from the solid-state imaging device by vertical streak correction.

Also, in the imaging system, the vertical streak correction switching unit switches on the vertical streak correction process while the timing of power supply is being adjusted by the AFE power-down controller.

According to the imaging system, noise can be reduced by the vertical streak correction process even while the timing of the AFE power-down is being adjusted.

Also, in the imaging system, the vertical streak correction switching unit switches on the vertical streak correction process when the timing adjustment by the AFE power-down controller has reached a limit.

A semiconductor integrated circuit according to the present invention includes an analog front-end (AFE) unit configured to process a captured-image signal from a solid-state imaging device, an image acquiring unit configured to acquire an image having a specific color from the AFE unit, a noise detector configured to detect noise of the specific-color image acquired by the image acquiring unit, and an AFE power-down controller configured to control timing of power supply to the AFE unit during a horizontal or vertical blanking period based on an amount of the noise detected by the noise detector.

### EFFECT OF THE INVENTION

According to the present invention, a control of reducing low-frequency noise is provided, and therefore, it is expected that image quality can be improved. Also, power supply to an AFE is adjusted to optimal timing (minimum noise), and therefore, it is expected that power consumption can be reduced.

### BRIEF DESCRIPTION OF THE DRAWINGS

FIG. 1 shows an overall configuration of an imaging system according to Embodiment 1.

FIG. 2(a) shows a range of AFE power-down, and FIG. 2(b) shows a timing chart.

FIG. 3 shows a mechanism of occurrence of low-frequency noise due to AFE power-down.

FIG. 4 shows a solution against low-frequency noise.

FIG. 5 shows a solution against low-frequency noise.

FIG. 6 shows a flowchart of the imaging system of Embodiment 1.

FIG. 7 shows a flowchart according to an imaging system according to Embodiment 2.

### DESCRIPTION OF THE REFERENCE CHARACTERS

101 image sensor
107 analog front-end (AFE)
109 vertical streak correction switching controller
110 black image acquisition controller
111 AFE power-down controller
112 noise detector

### BEST MODE FOR CARRYING OUT THE INVENTION

Hereinafter, the best mode for carrying out the present invention will be described with reference to the accompanying drawings.

### Embodiment 1

### Overall Configuration

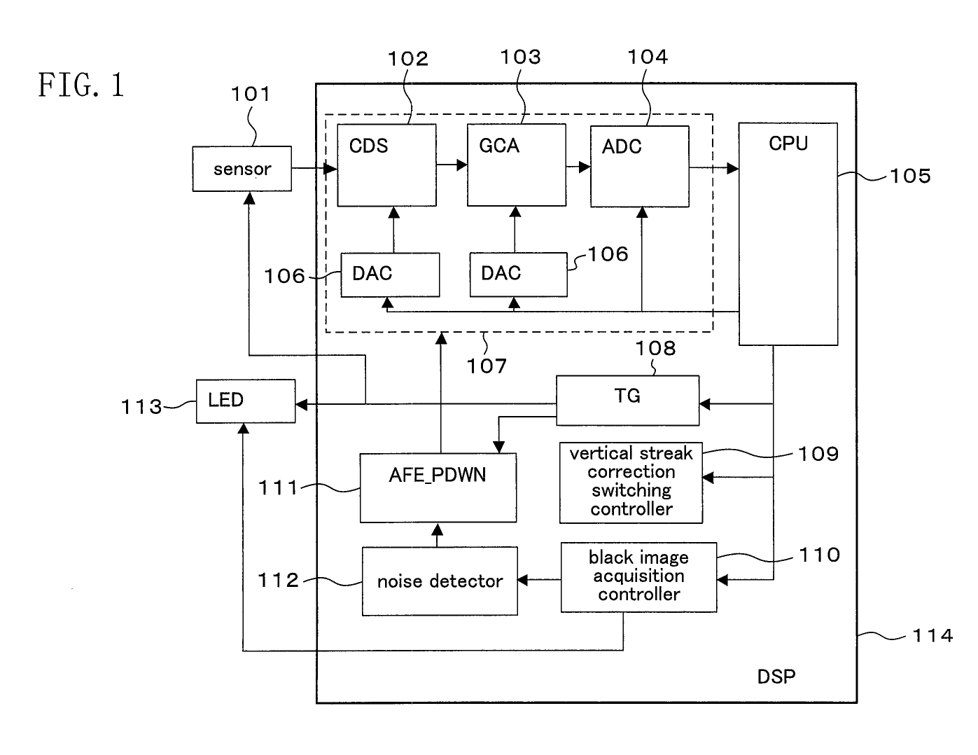

FIG. 1 shows an overall configuration of an imaging system according to Embodiment 1 of the present invention. The imaging system is assumed to be used as a medical capsule camera. The imaging system includes an image sensor 101, an LED 113, and a DSP 114.

The image sensor 101 is, for example, a solid-state imaging device, such as a CCD, a CMOS or the like. The image sensor 101 has a plurality of pixels. The pixels are provided in and around a valid pixel region which is used so as to capture an image of an object. The pixels provided around the valid pixel region are shielded from light.

The LED 113 is provided as lighting for capturing an image inside the body or the like.

The DSP 114 includes an Analog Front-End (AFE) 107, a CPU 105, a TG (Timing Generator) 108, a vertical streak correction switching controller 109, a black image acquisition controller 110, an AFE power-down controller (AFE_PDWN) 111, and a noise detector 112. Note that the DSP 114 may include a single chip (semiconductor integrated circuit) or a plurality of chips (semiconductor integrated circuits).

The AFE 107 subjects a captured-image signal (image data) output from the image sensor 101 to a predetermined process to convert the captured-image signal into a digital captured-image signal. The AFE 107 includes a CDS (Correlated Double Sampler) 102, a GCA (Gain-Controlled Amplifier) 103, an AD converter (Analog-to-Digital Converter) 104, and a DA converter (Digital-to-Analog Converter) 106. The CDS 102 performs correlated double sampling so as to remove amplifier noise and reset noise from a captured-image signal output from the image sensor 101. The GCA 103 amplifies a signal output from the CDS 102 by an adjustable gain. The AD converter 104 converts the signal amplified by the GCA 103 into a digital captured-image signal.

The CPU 105 controls the entire system. The TG 108 generates pulses which are used so as to capture an image. The pulses generated by the TG 108 are output to the image sensor 101 or the LED 113. The vertical streak correction switching controller 109 switches on/off vertical streak correction. The black image acquisition controller 110 acquires an image having a specific color (e.g., a black image in this example). The AFE power-down controller 111 controls timing of supplying power to the AFE 107.

[AFE Power-Down for Reduction of Power Consumption]

A conventional method (AFE power-down) for stopping power supply to an AFE during a vertical or horizontal blanking period so as to reduce power consumption has been proposed.

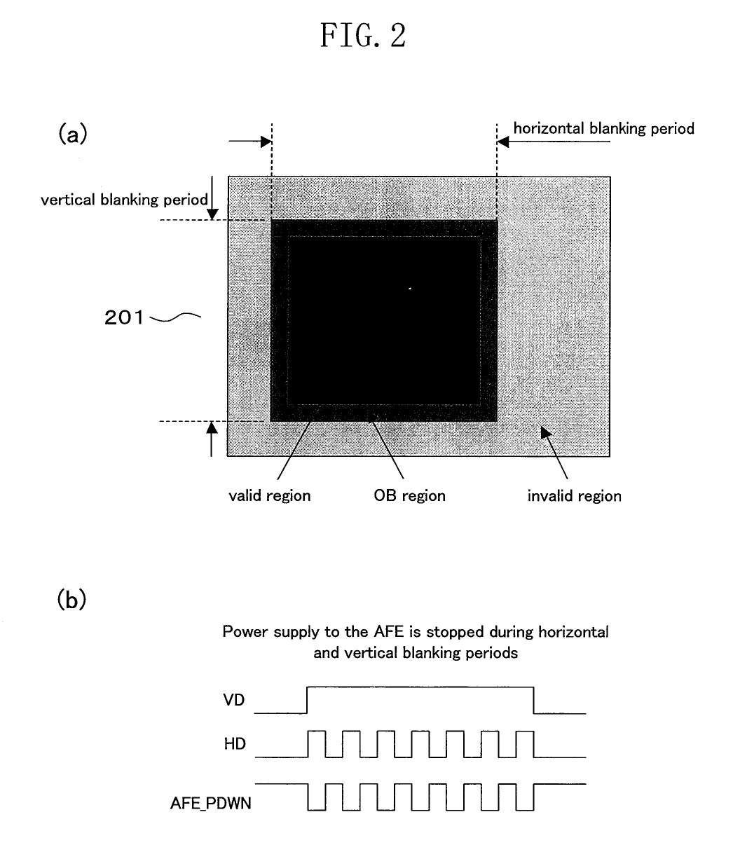

FIG. 2(a) shows a configuration of the image sensor 101 including a valid region, an invalid region, and an OB (Optical Black) region. An invalid region in a horizontal direction is referred to as horizontal blanking, and an invalid region in a vertical direction is referred to as vertical blanking.

FIG. 2(b) is a timing chart showing timings of VD indicating a vertical valid pixel region, HD indicating a horizontal valid pixel region, and AFE_PDWN indicating the presence or absence of power supply to the AFE. The power supply to the AFE is stopped (AFE_PDWN=‘H’) during a horizontal blanking period (HD=‘L’) and a vertical blanking period (VD=‘L’).

[Occurrence of Noise]

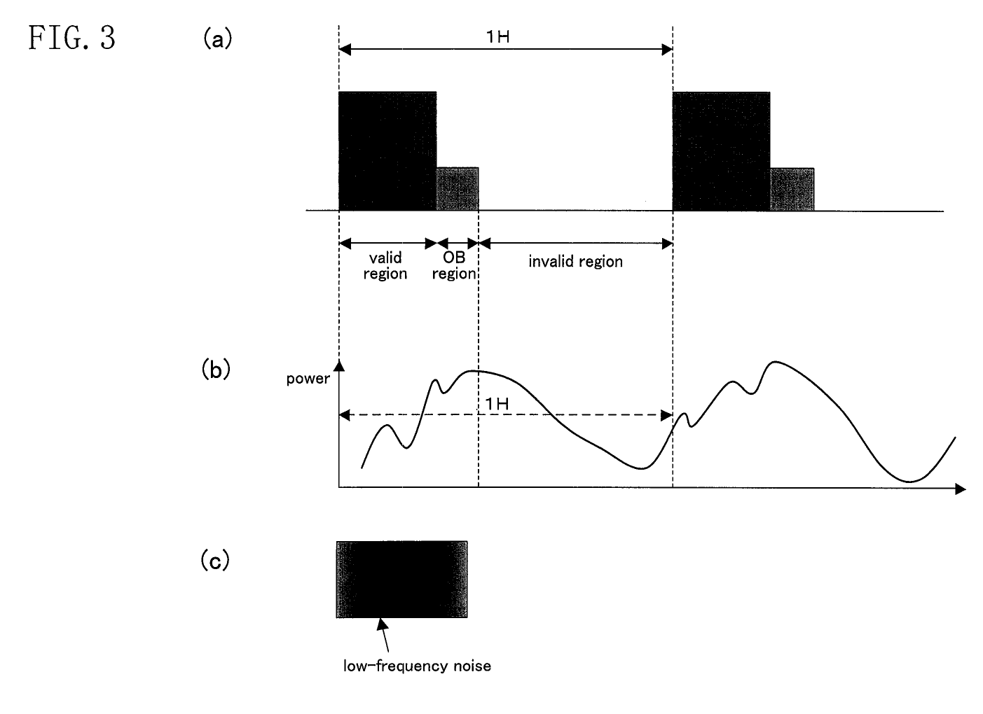

However, when the AFE power-down is performed during a horizontal blanking period, low-frequency noise may occur. FIG. 3 shows a mechanism of occurrence of noise due to the AFE power-down. FIG. 3(a) shows the valid region, the OB region and the invalid region for 1H. FIG. 3(b) shows how power is supplied to the AFE, corresponding to FIG. 3(a). When the AFE power-down is performed every 1H, a distortion may occur in a waveform of supplied power as shown in FIG. 3(b), depending on the performance of the AFE. When such a distortion occurs, low-frequency noise occurs as shown in FIG. 3(c).

[Solution]

Optimal timing which prevents noise is set at the factory before shipment. However, since a change in environment would cause the initially set timing to fail to prevent noise, a mechanism of dynamically adjusting the timing is required.

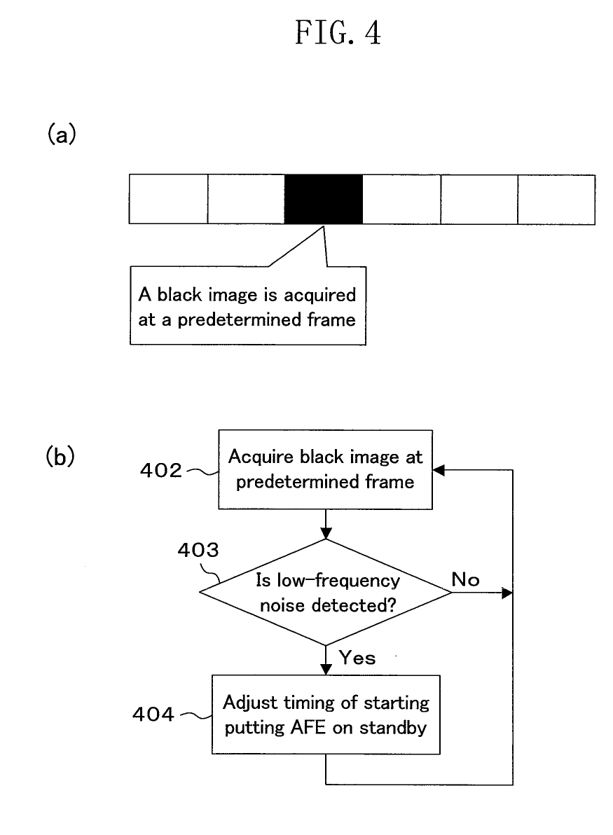

Low-frequency noise can be reduced by advancing the timing of resuming power supply in the AFE power-down. There is also conventional means for removing noise using vertical streak correction. FIG. 4 shows a method of acquiring a black image, determining noise, and adjusting the timing of the AFE power-down. FIG. 4(a) shows how a black image is acquired at a predetermined frame. A capsule camera or the like in the medical field has a control function of acquiring a black image by stopping light emission of an LED, and adjusting a black level. FIG. 4(b) shows a simple flowchart. In step 402, a black image is acquired at a predetermined frame. In step 403, it is determined whether low-frequency noise is present. Low-frequency noise would significantly appear in a black image. When low-frequency noise is detected, the timing of resuming power supply in the AFE power-down is adjusted in step 404.

The frequency of acquiring a black image can be set to a predetermined value by the user. Alternatively, the frequency can be automatically adjusted by determining the amount of noise.

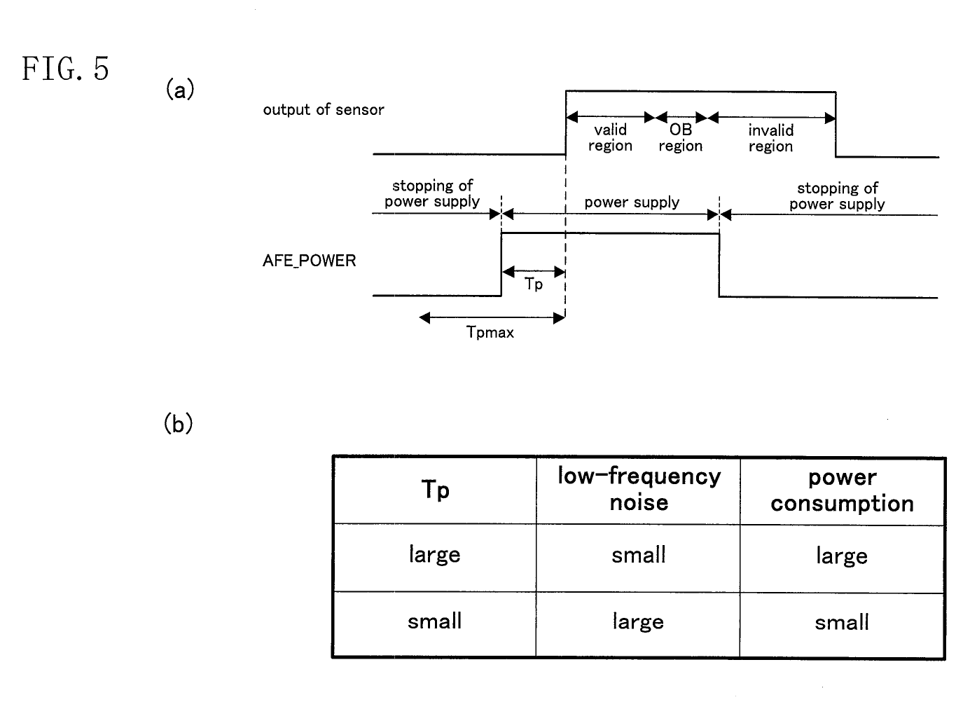

FIG. 5 shows a relationship between timing adjustment and power consumption. FIG. 5(a) is a timing chart showing the timing of the AFE power-down with respect to a sensor output (an output of the image sensor 101). Reference character Tp indicates a margin between the timing of resuming power supply in the AFE power-down and the timing of start of a valid region of the sensor output (blanking period end timing). In other words, power supply is resumed the time period Tp before the valid region start timing (blanking period end timing) of the sensor output. Reference character Tpmax indicates a maximum value of Tp. When Tp reaches Tpmax, the vertical streak correction process is switched on. There is a trade-off between the timing of resuming power supply in the AFE power-down and power consumption, which relationship is shown in FIG. 5(b). When low-frequency noise is detected, then if the position of Tp is shifted, power consumption can be efficiently reduced without occurrence of noise.

Thus, by automatically performing the adjustment by determining noise, differences between individual AFEs can be absorbed, and modification of a trial circuit can be flexibly dealt with. In addition, a capsule camera in the medical field can be corrected in response to a change in environment, such as a change in temperature or the like.

[Flowchart]

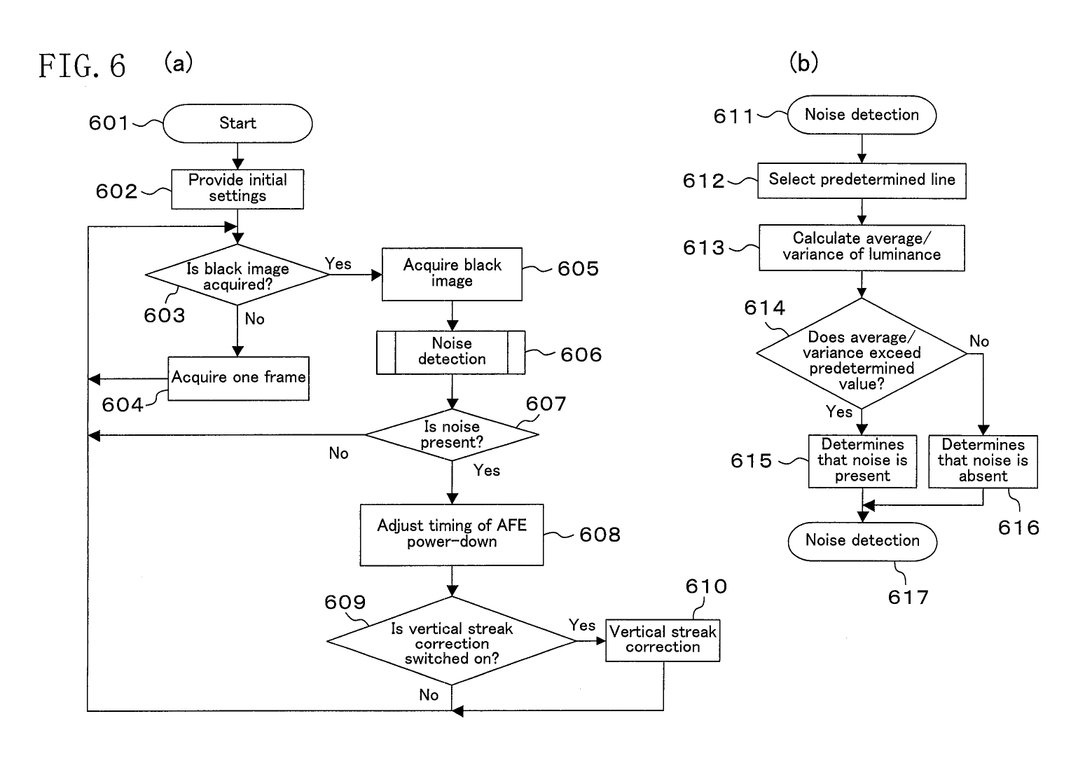

FIG. 6 shows a flow of the entire process.

When imaging is started (step 601), a clock is set and a sensor, a memory and the like are initially set (step 602). If imaging is started at a predetermined frame rate, a black image is acquired at predetermined cycles. In step 603, it is determined whether a black image has been acquired. When a black image has been acquired, control proceeds to step 605. Otherwise, control proceeds to step 604, in which the next frame is acquired, and thereafter, control returns to step 603.

The black image is acquired in step 605, and is subjected to noise detection in step 606. The noise detection will be described in detail below. In step 607, it is determined whether or not noise has been detected by the noise detection. When noise has been detected, control proceeds to step 608. Otherwise, control returns to step 603. In step 608, the timing of the AFE power-down is adjusted. Here, the timing of resuming power supply in the AFE power-down is advanced by a predetermined period of time since noise can be reduced by doing so.

It is not preferable that noise occur during the adjustment. In step 609, it is determined whether the vertical streak correction process is to be performed. If vertical streak correction is effective, control proceeds to step 610. Otherwise, control returns to step 603. Vertical streak correction is effective when the timing of the AFE power-down is being adjusted, and when the resumption timing Tp has reached Tpmax. When optimal timing is obtained, the vertical streak correction process is no longer effective. After step 610, control returns to step 603.

A flow of a process of the noise detection (step 606) are shown as steps 611 to 617. In step 612, a predetermined line of the valid pixel region is selected. A plurality of lines or all lines (a frame) may be selected. In step 613, either or both of an average and a variance of luminance are calculated. A value, such as a high-frequency component or the like, may be used instead of luminance. In step 614, it is determined whether a predetermined value is exceeded. If the predetermined value is exceeded, control proceeds to step 615. Otherwise, control proceeds to step 616.

[Noise Detection]

The noise detector 112 acquires an image having a specific color from the image acquiring unit 110 to perform detection. Low-frequency noise is significantly manifested by multiplying it by a gain, particularly in a black image. In this case, the presence or absence of noise is determined by calculating and comparing the variance or average of luminance with a predetermined value.

The speed of noise detection can be increased by subjecting pixels on a predetermined line(s) to noise detection instead of all pixels of one frame.

[Control of Switching On/Off Vertical Streak Correction]

Although noise is gradually reduced by dynamically adjusting the timing of the AFE power-down, it is not preferable that noise occur during the adjustment. Also, there is a limit of the adjustment, and if the AFE power-down is disabled, power consumption disadvantageously increases.

To avoid this, if noise is still detected while the timing of resuming power supply in the AFE power-down is being adjusted, the conventional vertical streak correction process is used to reduce noise. Also, if the adjustment has reached the limit (Tp has reached Tpmax), the adjustment of the timing of resuming power supply in the AFE power-down is switched to the vertical streak correction process.

### Embodiment 2

An imaging system according to Embodiment 2 of the present invention is assumed to be used as a digital still camera. The overall configuration of this imaging system is the same as that of FIG. 1, except that the LED 113 is removed. In the case of the capsule camera, the LED 113 is used as lighting in the body, which is not required for digital still cameras. As in Embodiment 1, the AFE power-down is performed so as to reduce power consumption, and noise occurs.

Digital still cameras are different from medical capsule cameras in that it is not preferable that a black image be acquired at predetermined frame intervals. Digital still cameras can acquire a black image during releasing for shooting, and therefore determines whether low-frequency noise has occurred every time shooting is performed. When determining that noise has occurred, the user is informed of the occurrence of the noise. The user determines whether to remove the noise. When noise is to be removed, noise removing means is used. In this case, however, since a black image is temporarily acquired, a normal image cannot be acquired.

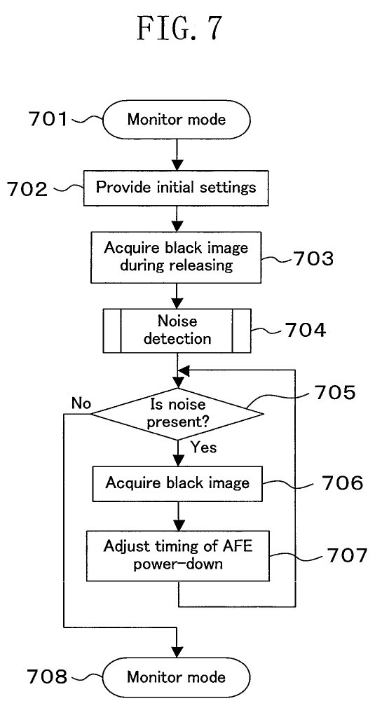

FIG. 7 shows a flowchart of Embodiment 2.

When shooting is started (step 701), a clock is set and a sensor, a memory and the like are initially set (step 702). Thereafter, the shutter is released for shooting from a monitor mode, and thereafter, a black image is acquired (step 703). In step 704, noise detection is performed. The detection method is similar to that of Embodiment 1. In step 705, it is determined whether noise is present. If noise is detected, control proceeds to step 706, and if otherwise, to step 708. In step 706, a black image is acquired. In step 707, the timing of the AFE power-down is adjusted. Steps 705 to 707 are repeatedly performed until noise is eliminated. After noise is eliminated, control proceeds to step 708, i.e., returns to the monitor mode.

Also in digital still cameras, power consumption can be reduced and the image quality can be improved, although the user needs to be informed, and taking a normal image is temporarily disabled.

### INDUSTRIAL APPLICABILITY

The imaging system of the present invention is applicable to a medical capsule camera, a digital still camera and the like.

1. An imaging system comprising:
a solid-state imaging device configured to convert light into an electrical signal to output a captured-image signal;
an analog front-end (AFE) unit configured to process the captured-image signal from the solid-state imaging device;
an image acquiring unit configured to acquire an image having a specific color from the AFE unit;
a noise detector configured to detect noise of the specific-color image acquired by the image acquiring unit; and
an AFE power-down controller configured to control timing of power supply to the AFE unit during a horizontal or vertical blanking period based on an amount of the noise detected by the noise detector.

2. The imaging system of claim 1, wherein
the image acquiring unit can acquire the specific-color image in any frame of the captured-image signal from the solid-state imaging device, and
the image acquiring unit adjusts a frequency of acquiring the specific-color image, depending on the amount of the noise detected by the noise detector.

3. The imaging system of claim 1, wherein
the noise detector determines the amount of the noise based on an average value of luminance of the specific-color image acquired by the image acquiring unit.

4. The imaging system of claim 1, wherein
the noise detector determines the amount of the noise based on a variance value of luminance of the specific-color image acquired by the image acquiring unit.

5. The imaging system of claim 1, wherein
the noise detector detects noise from a predetermined horizontal line.

6. The imaging system of claim 1, further comprising:
a vertical streak correction switching unit configured to switch on/off a process (vertical streak correction process) of removing noise from the captured-image signal output from the solid-state imaging device by vertical streak correction.

7. The imaging system of claim 6, wherein
the vertical streak correction switching unit switches on the vertical streak correction process while the timing of power supply is being adjusted by the AFE power-down controller.

8. The imaging system of claim 6, wherein
the vertical streak correction switching unit switches on the vertical streak correction process when the timing adjustment by the AFE power-down controller has reached a limit.

9. A semiconductor integrated circuit comprising:
an analog front-end (AFE) unit configured to process a captured-image signal from a solid-state imaging device;
an image acquiring unit configured to acquire an image having a specific color from the AFE unit;
a noise detector configured to detect noise of the specific-color image acquired by the image acquiring unit; and
an AFE power-down controller configured to control timing of power supply to the AFE unit during a horizontal or vertical blanking period based on an amount of the noise detected by the noise detector.
